(19) United States
(12) Patent Application Publication
(10) Pub. No.: US 2010/0129431 A1
(43) Pub. Date: May 27, 2010

(54) IDEBENONE COMPOSITION FOR THE TREATMENT OF NEUROLOGICAL DISORDERS

(75) Inventors: Joseph Schwarz, Markham (CA); Michael Weisspapir, Markham (CA)

Correspondence Address: ALPHARX INC., 168 KONRAD CRESCENT, SUITE 200, MARKHAM, L3R 9T9, CA

(73) Assignee: AlphaRx Inc., Markham (CA)

(21) Appl. No.: 12/276,809

(22) Filed: Nov. 24, 2008

(51) Int. Cl. A61K 9/127; A61K 31/122; A61P 31/00

(52) U.S. Cl. 424/450; 514/678

(57) ABSTRACT

The invention describes the use of an injectable form of Idebenone to induce protect from neuronal damage, improve recovery after brain trauma; stroke, intoxication, neurodegenerative diseases, memory loss or neuropathology associated with neuroinflammation or infection damage and to restore cognitive function, suppress disorientation, alcoholic and drug abuse associated syndromes and other signs of neuronal damage.

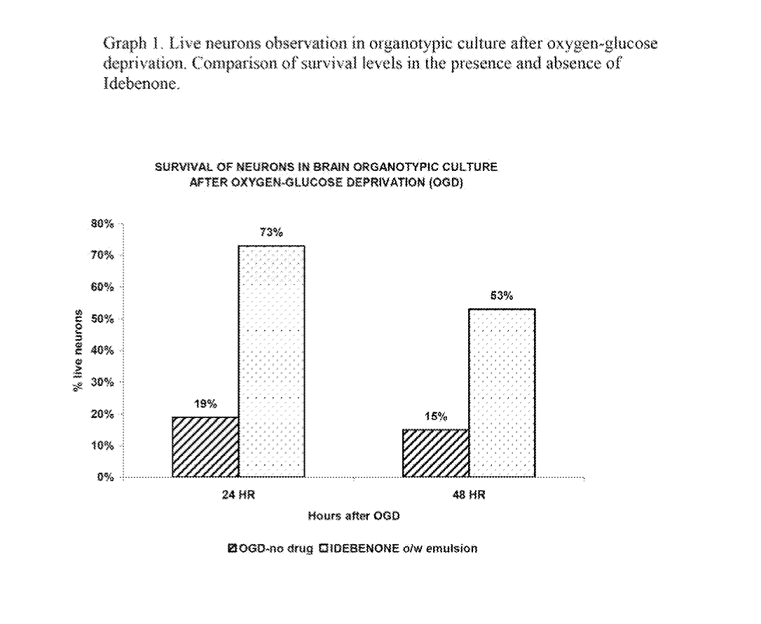

### FIELD OF INVENTION

The invention relates to the field of preparation of stable formulations of Idebenone, suitable for parenteral administration. Existing oral dosage forms of Idebenone associated with high metabolization in liver (“first pass effect”) can not be administered in an acute situation or in the case of patient unconsciousness. An injectable form of Idebenone would be in high demand.

### BACKGROUND OF THE INVENTION

Neuronal damage, neurodegenerative diseases and syndromes (Alzheimer disease, multiple sclerosis, Friedrich's ataxia, brain and spinal cord injury and neurotrauma, stroke, Parkinson disease, alcoholic intoxication and narcolepsy, post-operative and post-anesthesia recovery syndrome) and many other conditions require effective treatment and prophylaxis [1]

Post-Operative Stroke and Cognitive Deficit (POSCD) syndrome is common, especially in seniors undergoing extensive surgical procedures such as cardiac or hip-replacement surgery. There are over 2.5 million such surgical procedures undertaken annually in North America, with an incidence of POSCD of over 30%. There is a serious need for intervention and yet no adequate treatment options exist for this distressing post-surgical condition. In cardiac surgery alone, over 2 million surgical procedures are carried out annually in the US. Post anesthetic recovery from long-acting anesthetics often leaves patients in a state of marked disorientation and impaired cognitive function for prolonged periods of time. Even the advent of newer short-acting anesthetics does not alleviate the post-anesthetic effects on elderly surgical patients. [2]

The incidence of serious post-operative adverse events, including cognitive deficit and stroke, as high as 30-35% of extensive surgical procedures, results in extensive, prolonged hospital stays and serious quality of life issues for affected patients and their healthcare providers. The ability to reduce post-anesthetic stroke from ˜2.5% to 1.5%, or to reduce post-operative cognitive deficit substantially from the current ˜30% would result in significant cost-savings and improvements in quality of life management issues. There is now substantial evidence that many elderly patients experience cognitive deterioration postoperatively. In a prospective, randomized trial of general vs epidural anesthesia with sedation for total knee replacement in patients >70 yr of age, cognitive performance, as assessed with psychometric tests, was worse than the preoperative baseline in 4-6% of patients six months after anesthesia and surgery. Another large, prospective, controlled international study demonstrated a cognitive deficit in 9.9% of patients three months postoperatively whereas only about 3% of the age-matched controls were similarly impaired. Among patients over 75 yr of age, 14% had a persistent cognitive deficit after general anesthesia and surgery [2, 9].

In many cases, neurodegeneration associated with hypoxia is caused by decreased blood circulation and is accompanied by an excess of free radicals and the suppression of mitochondrial activity.

Antioxidants, are suggested as possible protective agents to diminish brain damage caused by extended general anesthesia. Various substances—antioxidants and radical scavengers—were tested in vitro in cell cultures, ex vivo in brain slices and in vivo in animal models. In such experiments, Idebenone, 2,3-dimethoxy-5-methyl-6-(10-hydroxydecyl)-1,4-benzoquinone demonstrated pronounced antioxidant activity and marked protection against oxidative damage to brain cells. An oral form of Idebenone is used in the treatment of cardiac muscle atrophy in Friedreich's Ataxia, as a liver protectant and, to a limited extent, in treatment of Alzheimer's disease [U.S. Pat. No. 5,916,925 “Pharmaceutical composition for treatment of dementia” and U.S. Pat. No. 6,133,322 Rustin P., et al. “Quinone derivatives for treating or preventing diseases associated with iron overload”]

In a small human study of nine patients with cerebrovascular disease, 90 mg Idebenone was given daily, and electroencephalograms and clinical symptoms, were monitored. The results suggested that Idebenone supplementation produced improvements in EEG and clinical symptoms in these patients [3].

Idebenone protects cultured cortical neurons against necrotic degeneration; it rescued cortical neurons even when applied 30 minutes after the NMDA pulse, suggesting that the drug interferes with the chain of toxic reactions triggered by an excessive stimulation of excitatory amino acid receptors [4]

Idebenone oral dosing (5 mg/kg daily for 8 weeks) in Friedreich's Ataxia patients significantly decreased a marker of oxidative DNA damage [5]. Idebenone prevented iron-induced lipo-peroxidation and cardiac muscle injury in three patients given 5 mg/kg daily for 4-9 months, resulting in a reduction of left ventricular enlargement in these patients. [6]

In experiments in cell cultures Idebenone scavenged a variety of free radical species [7]. Idebenone also redox coupled with hypervalent species of myoglobin or hemoglobin, thus preventing lipid peroxidation promoted by these species, Likewise, Idebenone has been shown to inhibit microsomal lipid peroxidation, induced by ADP-iron complexes or organic hydroperoxides. In so doing, Idebenone has been shown to prevent the destruction of cytochrome P450, which would otherwise accompany lipid peroxidation. It has been reported that Idebenone ameliorates learning and memory disturbances in experimental models produced by cerebral embolization, cerebral ischemia or lesions in the basal forebrain of rats, which is the area of origin of the acetylcholine neuron system projecting into the cerebral cortex, hippocampus and amygdala. In clinical tests, Idebenone was recognized to be effective in reducing psychological deficits such as a decline of memory retention and disorientation [8].

The bioavailability of oral Idebenone is relatively, high due to the polar hydrophobic nature of the molecule. However, oral administration of Idebenone is accompanied by a pronounced first pass metabolism in the liver and only small amounts of drug reach the brain or other targeted organs, as a result. Additionally, the effects of oral treatment only become apparent after several weeks or even months of drug use.

An injectable form of Idebenone would overcome a first pass effect of the oral dosage form and quickly provide the required concentration in blood and brain tissues. Nevertheless, no Idebenone dosage form suitable for parenteral delivery is currently available. The only described case of an intravenous administration of Idebenone in the medical literature was in a rat [8] experiment utilizing a 10% solution of polyethoxylated castor oil surfactant HCO-60, which is contraindicated for human use due to the extreme hemolytic properties of the vehicle.

The low water solubility of Idebenone makes the task of developing a parenteral form very difficult. The use of water miscible solvents (alcohol, propylene glycol, liquid PEG, N-methylpyrrolidone, etc.), where Idebenone dissolves well, is inappropriate due to the immediate precipitation of drug upon contact with physiological fluids or the water phase. An inclusion complex of Idebenone with cyclodextrin is described, but it is water dispersible, not soluble and not suitable for injection. The solubility of Idebenone in fixed oils (soy, corn, almond, etc.) is low; drug precipitates from such emulsions during storage, limiting their use for the preparation of emulsion based injectable forms of the drug.

### BRIEF DESCRIPTION OF THE INVENTION

An objective of the present invention is to provide an adequate method for the protection of brain cells from functional impairment caused by extended anesthesia, by using an injectable formulation of Idebenone. Surprisingly, it was found that a stable Idebenone formulation, suitable for parenteral administration, provided noticeable protection of brain tissues from cellular damage associated with hypoxic conditions. Such formulation was prepared by using oil-in water emulsion, made up of a mixture of distinct oily components. Idebenone concentration in the formulations varied from 0.1% to 2.5% by weight. The oil composition of the emulsion was compounded in such a manner that all incorporated Idebenone was completely dissolved in the discontinuous (oil) phase of the emulsion, avoiding drug precipitation during storage and providing a stable formulation. Formulations were administrated via intravenous, intraperitoneal or subcutaneous injections during in vivo tests, or added, after required dilution, to cell culture media during “in vitro” or “ex vivo” experiments, demonstrating excellent biocompatibility, absence of irritation or signs of toxicity and pronounced brain tissue protection.

The following examples are intended to illustrate certain preferred embodiments of the invention and no limitation upon the invention is implied by their inclusion.

### IDEBENONE FORMULATIONS

### Example 1-10

### Idebenone in Oil-in-Water Emulsions

### Example 1

### Preparation of Injectable Idebenone O/W Emulsion

Oil components of the formulation (Capric/caprylic triglycerides, acetylated monoglycerides and D-alpha-Tocopherol USP) were combined with lecithin and ethoxylated castor oil and mixed at 40° C. for 1 hour. Idebenone was dissolved in warm mixture of oils and surfactants and then blended with water phase, comprising water, EDTA and Glycerin using high shear rotor-stator mixer (5-10,000 rpm, 2 minutes). The obtained emulsion was treated with high pressure homogenizer (e.g., Avestin™ Emulsiflex C5) at 5,000-15,000 psi (300-1000 bar) for 3-5 cycles. After cooling to room temperature, the emulsion was filtered through sterile microporous membrane filter (0.2 mcm or 0.45 mcm) in aseptic conditions and dispensed into sterile glass vials. The sealed vials were stored in refrigerator or at room temperature, protected from light. The Idebenone content of the formulations was tested using HPLC method.

Examples 2-10 of Idebenone loaded o/w emulsions were prepared in similar manner, excluding example 8 where instead of high pressure homogenization the mixture of the oil and water phases was passed through 0.22 mcm microporous membrane, 3 times. Compositions of examples 1 through 10 are presented in table 1.

TABLE 1
Idebenone in oil-in-water emulsions (Examples 1-10)

| | 1 | 2 | 3 | 4 | 5 | 6 | 7 | 8 | 9 | 10 |
|---|---|---|---|---|---|---|---|---|---|---|
| | Percentage of composition | | | | | | | | | |
| Idebenone | 1.0 | 2.0 | 1.0 | 0.25 | 0.10 | 0.35 | 2.0 | 0.5 | 0.5 | 2.0 |
| Soya oil | | | 5.0 | 2.0 | | | | | | |
| Capric/caprylic | 12.5 | 18.0 | 10.0 | 8.0 | 10.0 | | 12.0 | | 16.0 | 18.0 |
| triglycerides (MCT) | | | | | | | | | | |
| Tocopherol USP | 0.1 | 8.0 | | | | | 3.0 | 2.5 | | 2.0 |
| Acetylated monoglycerides | 12.5 | | 10.0 | | | 10.0 | 15.0 | | 9.0 | 10.0 |
| Ethyl oleate | | | | | | | | 5.0 | | |
| Polysorbate-80 | | 1.0 | | | | | 0.5 | | 0.1 | 0.5 |
| TPGS | | | 0.5 | | | | | 1.0 | | |
| Ethoxylated castor oil | 0.5 | | | | 0.25 | | | | | |
| Lecithin USP | 2.0 | 1.5 | 1.8 | 1.0 | 1.0 | 2.0 | 1.5 | 1.0 | 1.5 | 1.5 |
| (phosphatidylcholine >70%) | | | | | | | | | | |
| Ethanol | | | 1.8 | | | | 1.5 | | | |
| Propylene glycol | | | | | | | | | | |
| Glycerin | 2.5 | 2.25 | | 2.25 | 2.25 | | | | 2.25 | 2.25 |
| Glycine | 0.5 | | | | 0.5 | | | 2.0 | | |
| EDTA | 0.02 | 0.02 | 0.02 | 0.02 | 0.02 | 0.02 | 0.02 | 0.02 | 0.02 | 0.02 |
| PURIFIED WATER to 100% | 68.38 | 67.23 | 69.88 | 86.48 | 85.88 | 87.63 | 64.48 | 87.98 | 70.63 | 63.73 |
| Total: | 100% | 100% | 100% | 100% | 100% | 100% | 100% | 100% | 100% | 100% |

Examples 11-16 of Idebenone loaded emulsion with increased content of oil phase were prepared either by high pressure homogenization or by spontaneous emulsification of Idebenone solution in mixture of the oil, surfactant and stabilizer after addition of water phase, without a homogenization step. For example 11, Idebenone was dissolved with slight heating (50-55° C.) in an oily mixture of acetylated monoglycerides (Myvacet™ 9-45K) and Vitamin E (Tocopherol mix), containing d-alpha tocopheryl polyethylene glycol 1000 succinate (Vitamin E TPGS) surfactant and soy lecithin. Propylene glycol was added to the warm solution, and then water phase, heated to 65-70° C., was added and mixed with oil composition using a propeller mixer at low speed to avoid foaming. Examples 13 and 15 were prepared in the same manner as example 11, while examples 12, 14 and 16 were treated with high pressure homogenizer. The formed oil-in-water emulsion was passed through microporous membrane filter (0.1 mcm) and stored at room temperature.

Compositions of examples 11 through 16 are presented in table 2.

TABLE 2
Idebenone in oil-in-water emulsions (Examples 11-16)
with high level of oil phase.

| | 11 | 12 | 13 | 14 | 15 | 16 |
|---|---|---|---|---|---|---|
| | Percentage of composition | | | | | |
| Idebenone | 1 | 2 | 2.5 | 2.5 | 2.5 | 2.5 |
| Soybean oil (LCT) | | 28 | 2 | 2 | 2 | |
| Capric/caprylic triglycerides | | | 14 | 8 | | |
| (MCT) | | | | | | |
| Tocopherol USP | 8 | | 8 | 4 | | 2 |
| Acetylated monoglycerides | 14 | | 8 | 16 | 16 | 16 |
| Triacetin | | | | | 10 | |
| Caprylic/Capric mono/di- | | | | | | 12 |
| glycerides | | | | | | |
| Oleic acid | | 0.05 | | | | |
| Polysorbate-80 | | 5 | | 4 | 4 | |
| Solutol ® HS-15 | | | | | | 5 |
| TPGS | 5 | | | | | |
| Ethoxylated castor oil | | | 5 | | | |
| (Incrocas-35) | | | | | | |
| Lecithin USP | 1.2 | 2 | 2.5 | 2 | 2 | 2.2 |
| (phosphatidylcholine >70%) | | | | | | |
| Ethanol | | 2 | 2.5 | | | |
| Propylene glycol | 5 | | 5 | | | 2.2 |
| Glycerin | | | | 2.25 | 2.25 | |
| Benzyl alcohol | | | | 0.5 | | |
| Dibasic sodium phosphate | | | | | 0.4 | 0.4 |
| EDTA disodium | 0.02 | 0.02 | 0.02 | 0.02 | 0.02 | 0.02 |
| Methyl paraben | 0.2 | 0.2 | 0.2 | | 0.2 | 0.2 |
| PURIFIED WATER to 100% | 65.58 | 60.73 | 40.28 | 58.73 | 60.63 | 57.48 |
| Total: | 100% | 100% | 100% | 100% | 100% | 100% |

Formulations 1-16 are stable at room temperature for several months with no signs of phase separation or Idebenone precipitation. The obtained oil-in-water emulsions were passed through microporous membrane filter (0.1 mcm) without loss of Idebenone content.

### Animal Experimental Protocol

Animals (Fisher 344 rats) were trained to perform memory testing tasks using a 12-limb radial maze device 24 hours before the start of the experimental procedures. The animals were divided into two groups: a control group that was injected with vehicle and experimental group, treated with injectable Idebenone. All animals were anesthetized to a certain level with the inhalational anesthetic isoflurane for one hour without any surgical intervention. Cognitive behavior and memory testing were carried out to determine the post anesthesia change in locomotor activity and choice accuracy in working memory tasks at 4, 24 and 48 hours post-anesthesia.

The experimental animals were given Idebenone intraperitoneally in dose of 10 mg/kg 15 minutes before anesthesia. Observations confirmed a significant improvement in cognitive function and working memory in the test group compared with control group for all observed time points. Awakening time after general anesthesia substantially decreased for animals given Idebenone injection.

### Oxygen-Glucose Deprivation in Acute and Organotypic Hyppocampal Slices

The neuroprotective effects of Idebenone were evaluated in hippocampal slice cultures, which enabled relatively long-term assessment of the survival of several different neuronal populations. Hippocampal slices were prepared from 14-20 day old Wistar rats after the entire hippocampus was removed and slices (400 μm) were made with a Mac-Ilwain tissue chopper. Cell viability was assessed by the control of electrophysiological activity.

Organotypic slices (250-300 mcm thickness) were grown using standard methods, then transferred onto 30-mm diameter membrane inserts and put into 6-well culture trays, with 1.5 mL of slice culture medium per well. Slices were kept in culture for 7-14 days before the study. In vitro ischemia was simulated by hypoxia combined with a glucose-free medium (Oxygen-Glucose Deprivation, OGD). Before hypoxia, the slices were washed three times with glucose-free Hank's balanced salt solution (HBSS). The cultures were then placed into an airtight Incubator chamber through which 95% N2/5% CO₂ gas preheated to 37° C. was passed at 5-10 L per minute. The temperature of the chamber was kept at 37° C. and the partial pressure of oxygen was approximately 0-0.2 mm Hg. For the studies involving Idebenone formulations, slices were rinsed with glucose-free HBSS containing Idebenone in submicron emulsion, diluted to obtain 0.1 or 1.0 μM concentrations in culture; the drug remained in contact with the cultures for the duration of the OGD. After the insult, the culture tray was removed from the chamber, the anoxic-glucose-free HBSS was aspirated from the wells, and standard (oxygenated) slice culture media was added. Cell viability was assessed by propidium iodide staining. The use of Idebenone in submicron emulsion (SME) increased survival time in acute hippocampal slices for more than 50%, from 7.0 to 10.6 minutes (see Table 3)

TABLE 3
Comparative time of electrophysiological activity persisting
in acute hippocampal slices in OGD

| | Electrophysiological |
|---|---|
| Test article | activity in OGD |
| Control vehicle (diluted o/w emulsion) | 7.0 minutes |
| Idebenone 1 micromol/L in o/w | 10.6 minutes |
| emulsion (example 6) | |

The administration of an injectable form of Idebenone increased the survival of neurons in brain organotypic culture slices after OGD several times when compared to control (see Graph 1)

1. A method of treatment of neurological disorders and improving conditions associated with neuronal damage by parenteral administration of a neuroprotective pharmaceutical composition, comprising of at least one physiologically acceptable derivative of 1,4-benzoquinone.

2. A method as set forth in claim 1 wherein said derivative is 1,4-benzoquinone is 2,3-dimethoxy-5-methyl-6-(10-hydroxydecyl)-1,4-benzoquinone (Idebenone).

3. A method as set forth in claim 1 wherein said composition is administrated parenterally via intravenous injection, intravenous infusion, intra-arterial, intramuscular, subcutaneous or intraperitoneal injection.

4. A method as set forth in claim 1 wherein said derivative of 1,4-benzoquinone is administrated in dosage concentrations of 0.5 to 50 mg/kg per day.

5. A method as set forth in claim 1 wherein said composition is a colloidal delivery system, elected from micellar preparations, emulsions, solid lipid nanoparticles, polymeric anoparticles, nanocapsules or suspensions, wherein said 1,4-benzoquinone derivative is associated with the hydrophobic phase of the colloidal system.

6. A method as set forth in claim 5 wherein said emulsion is an oil-in-water emulsion.

7. A method as set forth in claim 1 wherein said neurological disorder is caused by Neurotrauma.

8. A method as set forth in claim 1 wherein said neurological disorder is stroke.

9. A method as set forth in claim 1 wherein said neurological disorder is encephalomyelitis.

10. A method as set forth in claim 1 wherein said neurological disorder is associated with mitochondrial dysfunction.

11. A method as set forth in claim 1 wherein said neurological disorder is associated with an hypoxic condition.

12. A method as set forth in claim 1 wherein said neurological disorder is alcohol withdrawal syndrome or fetal alcoholic syndrome.

13. A method as set forth in claim 1 wherein said neurological disorder is associated with neuronal damage caused by bacterial or viral infection.
